(19) United States
(12) Patent Application Publication
(10) Pub. No.: US 2026/0263296 A1
(43) Pub. Date: Sep. 10, 2026

(54) TWO-PARTS ABSORBENT ARTICLE

(71) Applicant: The Procter & Gamble Company, Cincinnati, OH (US)

(72) Inventors: Wei DAI, Beijing (CN); Ling TONG, Beijing (CN); Jing ZHANG, Beijing (CN); Sarah Marie WADE, Cincinnati, OH (US)

(21) Appl. No.: 19/668,179

(22) Filed: May 5, 2026

Related U.S. Application Data
(63) Continuation of application No. PCT/CN2024/122300, filed on Sep. 29, 2024.

(30) Foreign Application Priority Data
Nov. 10, 2023 (WO) PCT/CN2023/131003
Aug. 9, 2024 (WO) PCT/CN2024/110974

(51) Int. Cl. A61F 13/70; A61F 13/49; A61F 13/496; A61F 13/56; A61F 13/72; A61F 13/84

(52) U.S. Cl. CPC A61F 13/70; A61F 13/496; A61F 13/565; A61F 13/72; A61F 2013/49063; A61F 2013/5672; A61F 2013/5677; A61F 2013/8479

(57) ABSTRACT

Disclosed is a pant absorbent article having a belt part and a crotch part, the belt part and the crotch part being detachable with each other by a primary fastening system for providing the absorbent article. The belt part includes a front belt and a back belt, the belt part having elasticity in the transverse direction and having a pair of side seams having a seam length LS. The front belt and the back belt each have a primary belt fastening means on a garment facing side, the primary belt fastening means forming part of the fastening system engageable with a primary crotch fastening means.

### CROSS REFERENCE TO RELATED APPLICATION

This application is a continuation of, and claims priority under 35 U.S.C. § 120 to, PCT Patent Application PCT/CN2024/122300, filed on Sep. 29, 2024, which claims priority, under 35 U.S.C. § 119, to PCT Patent Application PCT/CN2024/110974, filed Aug. 9, 2024, and PCT Patent Application PCT/CN2023/131003, filed on Nov. 10, 2023, the entire disclosures of all of which are hereby incorporated by reference herein.

### FIELD OF THE INVENTION

This invention relates to a two-parts absorbent article, namely a belt part and a crotch part which, when assembled with each other, provides a pant type absorbent article. The crotch part may be exchanged after soiling to make use of the belt part for more than one usage.

### BACKGROUND OF THE INVENTION

Absorbent articles for personal hygiene, such as disposable taped diapers, disposable pants, and adult incontinence undergarments, are designed to absorb and contain various body exudates, including urine, menses, and fecal matter. Absorbent articles capable of exchanging absorbent bodies which enables a second usage after the first soiling are desired. Such absorbent article may be actualized by providing a crotch part attachable to a belt part, wherein the crotch part may be removed after the first soiling, and thereby enabling exchange with another crotch part. Such absorbent having multiple absorbent bodies may provide convenience by enabling removal of the soiled absorbent body without complete removal of garments. Such convenience may be particularly desired during outing from the home, or during cold seasons. Absorbent articles having exchangeable crotch parts may also provide conservation of material.

Absorbent articles having exchangeable crotch parts have been known in the art, such as in Japanese Patent Publications 2023-94947A-94954A and 2023-94956A-94958A. While such absorbent articles have been known, they may require improvements for actual usage.

For example, those known in the art may not have measures for assembling the parts securely in the intended position against each other, to provide the resulting pant article to have fundamental functions such as containment, leakage prevention, or wear comfort. For example, those known in the art may not have practical measures for providing the belt part in a reusable form. For example, those known in the art may not have practical measures for providing the fastening system for the two-parts absorbent article in a reusable form.

Based on the foregoing, there is a need for an absorbent article having two parts which provide easy and reliable application and attachment with each other. There is also a need for such absorbent article which provides fundamental functions such as good containment, leakage prevention, and wear comfort. There is also a need for such an absorbent article which addresses the concerns of consumers who are conscious about sustainable usage of material, while also providing an aesthetically acceptable appearance.

### SUMMARY OF THE INVENTION

The present invention is directed to a pant absorbent article comprising a belt part and a crotch part, the belt part and the crotch part being detachable with each other by a primary fastening system for providing the absorbent article, the absorbent article having a longitudinal direction and a transverse direction, the belt part having a front belt and a back belt, the belt part having elasticity in the transverse direction and comprising a pair of side seams having a seam length LS, the front belt and the back belt each comprising a primary belt fastening means on the garment facing side, the primary belt fastening means forming part of the fastening system engageable with a primary crotch fastening means;

the crotch part comprising a water permeable topsheet, a water impermeable backsheet, an absorbent core disposed between the topsheet and the backsheet, and the primary crotch fastening means disposed on the wearer facing side, wherein the primary belt fastening means and the primary crotch fastening means are fastened magnetically.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIGS. 1A-1F are schematic perspective views of embodiments of the two-part absorbent article of the present invention showing how the belt part and the crotch part are attached with each other to form a pant-type absorbent article.

FIGS. 2A-2D are schematic plan views of embodiments of the front belt of the present invention with any seam removed and in a flat uncontracted condition showing the garment facing surface.

FIGS. 3A-3D are schematic plan views of embodiments of the back belt of the present invention with any seam removed and in a flat uncontracted condition showing the garment facing surface.

FIGS. 4A-4B are schematic cross section views of FIGS. 2A-3A taken along lines 4A and 4B.

FIGS. 5A-5C are schematic plan views of embodiments of the crotch part of the present invention in a flat uncontracted condition showing the wearer facing surface.

FIG. 6 is a schematic cross section views of FIG. 5A taken along line 6.

FIGS. 7A-7B are schematic cross section views of FIG. 5C taken along line 7.

FIG. 8A is a schematic plan view of the belt part of FIGS. 2A, 3A, and the crotch part of FIG. 5A assembled into a pant absorbent article.

FIG. 8B is a schematic plan view of the belt part of FIGS. 2C-2D, 3C-3D and the crotch part of FIG. 5C assembled into a pant absorbent article.

FIGS. 9A-9E are perspective and transverse schematic cross section views of embodiments of the magnet piece pair of opposite polarity of the present invention.

FIG. 10B is a transverse schematic cross section view of FIG. 8B taken along lines 10B, and FIG. 10A is FIG. 10B with the straps in the open configuration.

FIGS. 11A1-11C2 are perspective and transverse schematic cross section views of an embodiment of the primary fastening means of the present invention.

FIG. 12 is a schematic view of an example of a hanger-type sample holding fixture according to the “Whole Article Force Measurement” herein.

FIGS. 13A-13C are schematic views of an example of equipment according to the “Bending Force Measurement” herein.

FIG. 14A is a schematic view of how to measure the “Angle of Concave Notch” herein.

FIG. 14B is a schematic view of how to measure the dimensions of a secondary fastening means herein.

### DEFINITIONS

As used herein, the following terms shall have the meaning specified thereafter:

“Absorbent article” refers to articles of wear which may be in the form of pants, taped diapers, incontinent briefs, feminine hygiene garments, and the like, so configured to also absorb and contain various exudates such as urine, feces, and menses discharged from the body. The “absorbent article” may serve as an outer cover adaptable to be joined with a separable disposable absorbent insert for providing absorbent and containment function, such as those disclosed in PCT publication WO 2011/087503A.

“Pant” refers to disposable absorbent articles having a pre-formed waist and leg opening 35s. A pant may be donned by inserting a wearer's legs into the leg opening 35s and sliding the pant into position about the wearer's lower torso. Pants are also commonly referred to as “closed diapers”, “prefastened diapers”, “pull-on diapers”, “training pants” and “diaper-pants”.

“Longitudinal” refers to a direction running substantially perpendicular from a waist edge to an opposing waist edge of the article and generally parallel to the maximum linear dimension of the pant type absorbent article when the belt part and the crotch part are attached with each other.

“Transverse” refers to a direction perpendicular to the longitudinal direction.

“Proximal” and “distal” refer respectively to the position closer or farther relative to the longitudinal center of the pant type absorbent article when the belt part and the crotch part are attached with each other.

“Wearer-facing” and “garment-facing” refer respectively to the relative location of an element or a surface of an element or group of elements of the pant type absorbent article when the belt part and the crotch part are attached with each other. “Wearer-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the diaper article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate member(s) which in turn are affixed to the other element.

“Film” refers to a sheet-like material wherein the length and width of the material far exceed the thickness of the material. Typically, films have a thickness of about 0.5 mm or less.

“Nonwoven”, nonwoven layer” or “nonwoven web” are used interchangeably to mean an engineered fibrous assembly, primarily planar, which has been given a designed level of structural integrity by physical and/or chemical means, excluding weaving, knitting or papermaking (ISO 9092:2019 definition). The directionally or randomly orientated fibers, are bonded by friction, and/or cohesion and/or adhesion. The fibers may be of natural or synthetic origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yam). Nonwoven webs can be formed by many processes such as meltblowing, spunbonding, solvent spinning, electrospinning, carding and airlaying. The basis weight of nonwoven webs is usually expressed in grams per square meter (g/m² or gsm).

“Water-permeable” and “water-impermeable” refer to the penetrability of materials in the context of the intended usage of disposable diaper articles. Specifically, the term “water-permeable” refers to a layer or a layered structure having pores, opening 35s, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Hydrophilic” describes surfaces of substrates which are wettable by aqueous fluids (e.g., aqueous body fluids) deposited on these substrates. Hydrophilicity and wettability are typically defined in terms of contact angle and the strike-through time of the fluids, for example through a nonwoven fabric. This is discussed in detail in the American Chemical Society publication entitled “Contact Angle, Wettability and Adhesion”, edited by Robert F. Gould (Copyright 1964). A surface of a substrate is said to be wetted by a fluid (i.e., hydrophilic) when either the contact angle between the fluid and the surface is less than 90°, or when the fluid tends to spread spontaneously across the surface of the substrate, both conditions are normally co-existing. Conversely, a substrate is considered to be “hydrophobic” if the contact angle is greater than 90° and the fluid does not spread spontaneously across the surface of the fiber.

“Extendibility” and “extensible” mean that the width or length of the component in a relaxed state can be extended or increased.

“Elasticated” and “elasticized” mean that a component comprises at least a portion made of elastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Dimension”, “Length”, “Width”, “Pitch”, “Diameter”, “Aspect Ratio”, “Angle”, and “Area” of the article are all measured in a state wherein the article is extended to the Full Stretch Circumference W1 according to the “Whole Article Force Measurement” herein, and utilizing a ruler or a loupe, unless specified otherwise.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0±2, and a b* value equal to 0±2 according to the CIE L*a*b* color system.

### DETAILED DESCRIPTION OF THE INVENTION

### Two-Parts Absorbent Article

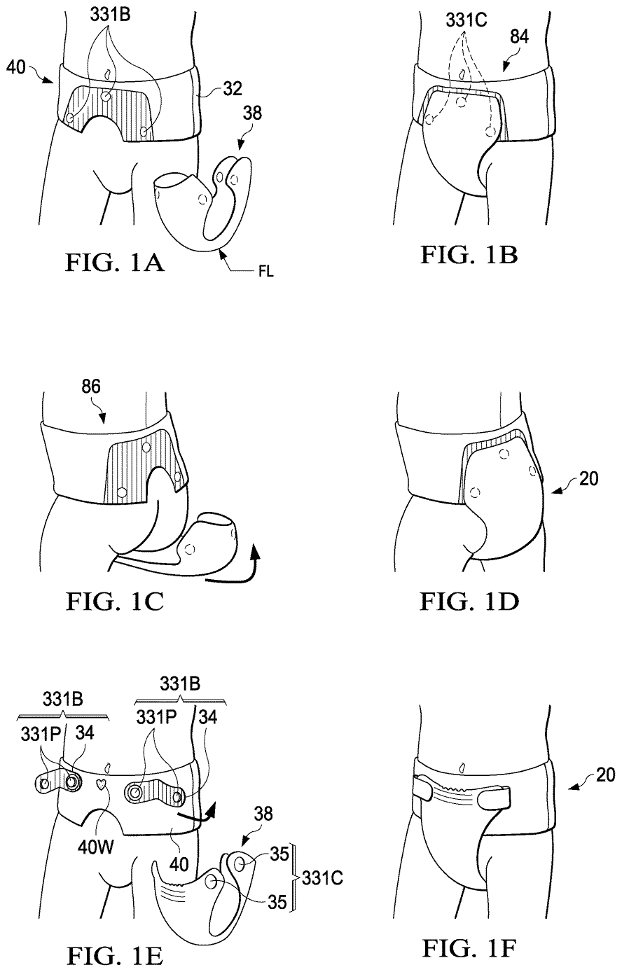

FIGS. 1A-1F are perspective views of embodiments of a two-parts absorbent article of the present invention made of a belt part (40) and a crotch part (38) which, when assembled with each other, forms a pant absorbent article (20). The belt part (40) and the crotch part (38) are detachable with each other by a primary belt fastening means (331B) and a primary crotch fastening means (331C) fastened magnetically. Each of the belt part (40), the crotch part (38), and the pant type absorbent article (20) formed by assembling the belt part (40) and the crotch part (38) have a longitudinal direction and a transverse direction. Each of the belt part (40), the crotch part (38), and the pant type absorbent article (20) have a longitudinal centerline LX which also serves as the longitudinal axis. Each of the belt part (40) and the crotch part (38) have a transverse centerline TX which also serves as the transverse axis. The two-parts absorbent article of the present invention may be provided in various forms of merchandize or commercially available goods associated with the two-parts absorbent article, for example as a kit including necessary parts for providing the pant absorbent article, or one of the parts including size information and methods of attachment with the other part for enabling the purchaser to match with the corresponding merchandize for forming the pant absorbent article.

The belt part (40) is formed to fit the torso circumference of the intended wearer to dynamically create fitment forces and to distribute the forces dynamically generated during wear. The belt part (40) has a front belt (84) and a back belt (86), the belt part (40) having elasticity in the transverse direction and comprising a pair of side seams having a seam length LS, wherein the front belt (84) and the back belt (86) form a ring-like elastic part. The front and back belts (84, 86) may be imparted elasticity by elastic bodies, for example elastic films and elastic strands laminated between nonwoven substrates. In such embodiment, the front and back belts are typically bonded together to form left and right side seams (32). Side seam bonding may be provided by heat, heated air, ultrasonic energy, pressure, adhesive, or the combination thereof. The front and back belts (84, 86) may be durable material, as described in further detail below, imparted elasticity by elastic bodies laminated between or attached on the surface of the fabrics, or by weaving or knitting of threads forming the fabrics. Regardless of the material forming the front and back belts (84, 86), the transverse edges dividing the front region and back region of the belt part (84, 86) are considered the side seams (32). When the belt part (40) is homogeneous in the circumferential direction, any 2 lines that are on the farthest sides in the transverse direction are considered the side seams.

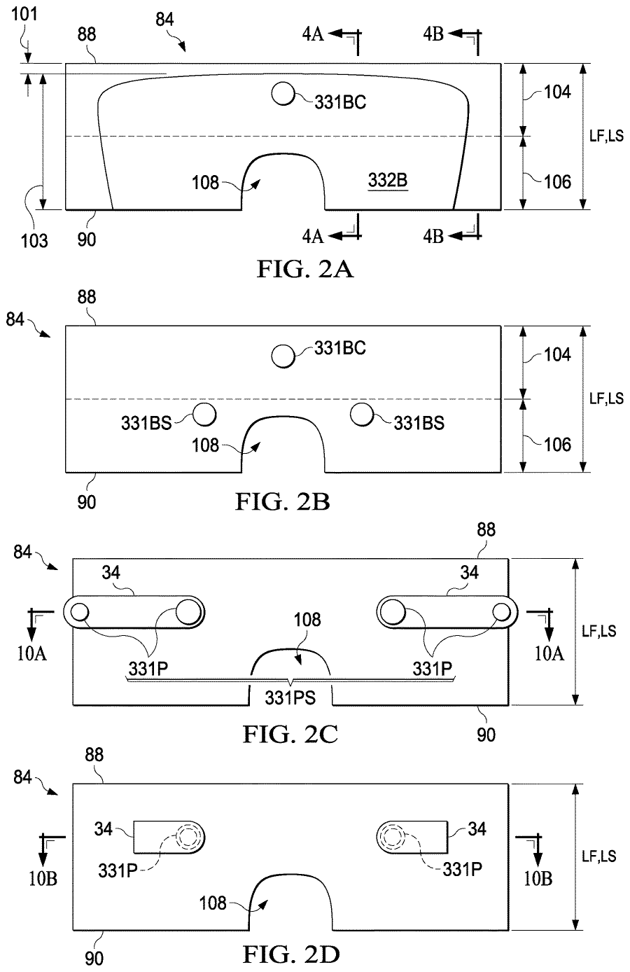

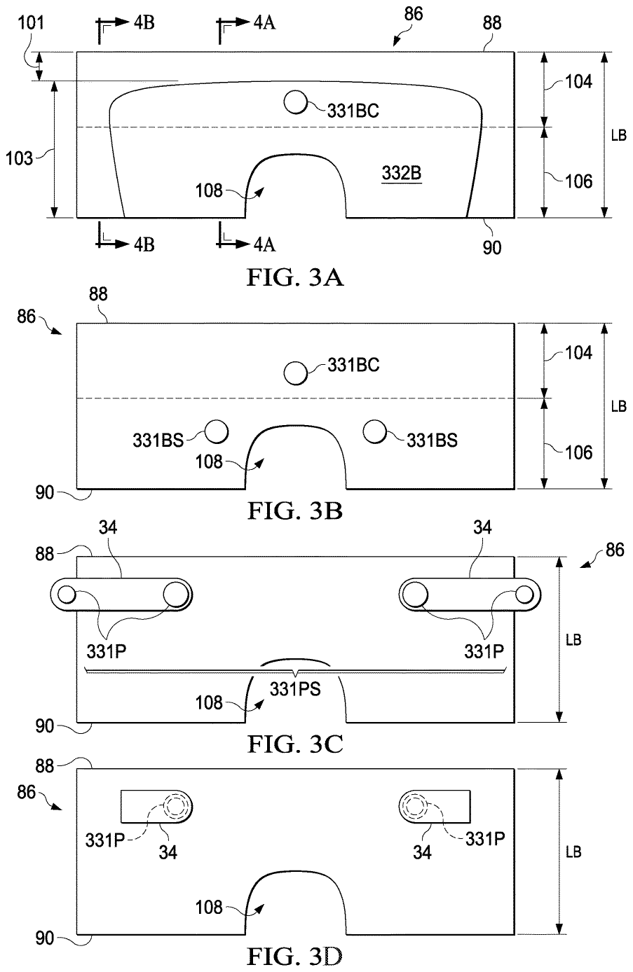

The longitudinal dimension of the belt part (40) along its circumference may be consistent, or may be varied, as described in further detail below. Referring to FIGS. 2A-3D, the back belt (86) may have a greater maximum longitudinal dimension than that of the front belt (84). When the front belt (84) has a maximum longitudinal dimension LF and the back belt (86) has a longitudinal dimension LB, LB may be from about 105% to about 130% of LF. Without being bound by theory, such dimension difference between the front and back belt (84. 86) may allow easy distinction between front and back, and further provide better fit for the buttock. In such embodiment, the distal edges of the front and back belt (84, 86) may be matched with each other to form a waist opening, wherein the remaining dimension of the back belt forms an appendix portion functioning as a buttock cover. Still referring to FIGS. 2A-3D, the front belt (84) and the back belt (86) is each divided into multiple zones spanning in the transverse direction and defined by its location from the distal edge (88) to the proximal edge (90) relative to the percentage of the seam length LS wherein the distal edge (88) is considered 0% and the proximal edge (90) is considered 100%. The front belt (84) and the back belt (86) each comprises a primary belt fastening means (331B) on the garment facing side, the primary belt fastening means (331B) forming part of the fastening system (33) engageable with the primary crotch fastening means (331C). The position of the primary belt fastening means (331B) and any other fastening means such as the secondary fastening means (332B) as explained below, are collectively considered the belt fastening region, and the belt fastening region relative to the seam length LS is considered the fastening zone (103). When provided as a merchandize, the belt part (40) may be in a contracted and flattened state with the side seams (32) positioned at the left and right ends. The belt parts (40) may then optionally be folded at one or more lines approximately parallel to the longitudinal axis or the transverse axis. Alternatively, the belt parts (40) may optionally be rolled up in the transverse direction to provide a near cylindrical shape. The belt parts (40) in such state may then be stacked up in the thickness direction or compiled with each other and packaged in a primary packaging consisting of only the belt parts (40).

The crotch part (38) is formed to fit the crotch of the intended wearer to absorb and contain bodily exudates while also creating fitment forces during wear. Referring to FIGS. 1A-1F and 6, the crotch part (38) comprises a water permeable topsheet (24), a water impermeable backsheet (25), an absorbent core (62) disposed between the topsheet (24) and the backsheet (25), and a primary crotch fastening means (331C) disposed on the wearer facing side. After soiling, the crotch part (38) may be detached and exchanged with a fresh crotch part (38) to make use of the belt part (40) for more than one usage. Such exchange of the crotch part (38) also enables diaper change without removing the entire absorbent article (20) from the wearer.

When provided as a merchandize, the crotch part (38) may be provided in a folded state, wherein the folded state is effected by folding the crotch part (38) along a folding line (FL) parallel to the longitudinal axis LX. The folding line (FL) may be just one and match the longitudinal axis LX, or may be two and not matching, but parallel to the longitudinal axis LX. By providing the crotch part (38) in such folded state prior to attaching with the belt part (40), the crotch part (38) may be effectively inserted between the wearer's legs and attached to the belt part (40). The crotch parts (38) may then optionally be folded at one or more lines approximately parallel to the transverse axis. Alternatively, the crotch parts (38) may then optionally be rolled up in the transverse direction to provide a near cylindrical shape. The crotch parts (38) in such state then may be stacked up in the thickness direction or compiled with each other and packaged in a primary packaging consisting of only the crotch parts (38).

Referring to FIGS. 1A-1F, the pant absorbent article (20) the present invention may be assembled by the steps of:
1) donning the belt part (40) on the torso of the wearer;
2) inserting the crotch part (38) between the legs of the wearer;
3) opening the folded state of the crotch part (38) to expose the primary crotch fastening means (331C); and
4) attaching the primary crotch fastening means (331C) to the primary belt fastening means (331B).

Referring to FIG. 1E, the belt part (40) may comprise a wearing belt indicia (40W) for positioning the belt part in the intended position relative to a wearer upon donning the belt part (40) on the torso of the wearer. The wearing belt indicia (40W) may be provided adjacent the distal edge on the longitudinal axis in order to match with the navel of the wearer. Alternatively or additionally, the wearing belt indicia (40W) may be provided in plurality adjacent the distal edge of the side seams (32) in order to match with the iliac crests of the wearer. Referring to FIGS. 1A-1D, the belt part (40) may further comprise a fastening belt indicia, and the crotch part (38) may comprise a fastening crotch indicia, wherein the fastening belt indicia and the fastening crotch indicia are so configured to assist attachment of the belt part (40) and the crotch part (38) in the intended position. The primary crotch fastening means (331C) and the primary belt fastening means (331B) themselves may serve as the fastening belt indicia and the fastening crotch indicia, as explained in further detail below. Referring to FIG. 1F, the wearing belt indicia (40W) may be so configured that, when the belt part (40) and crotch part (38) are assembled in the intended position, the wearing belt indicia (40W) is no longer seen, thus indicating successful assembly of the pant-type absorbent article (20).

A primary packaging consisting of a first plurality of belt parts (40) and another primary packaging consisting of a second plurality of crotch parts (38) may be packaged together in a secondary packaging to provide a kit merchandize. In such kit merchandize, the second plurality may be greater than the first plurality. Alternatively, a packaging consisting of only the belt parts (40) or crotch parts (38) may be provided a merchandize, wherein the merchandize includes size information and methods of attachment for enabling the purchaser to match with the corresponding size of the belt part (40) or crotch part (38) to form the pant-type absorbent article (20). Alternatively, the kit merchandize may consist of one or more belt parts (40) and one or more crotch parts (38) in the same primary packaging for use as, for example, a travel kit, sample, gift, or otherwise.

The belt part (40) and the crotch part (38) may be so configured to provide a one-to-one matching of the same size. Alternatively, there may be provided variation of crotch parts (38) that match the same belt part (40). For example, crotch parts (38) of different containment capacity may be offered for day time use and night time use. For example, different sizes of the belt part (40) and the crotch part (38) may be matched in order to fit unique body construction of the wearer.

As described above, the crotch part (38) of the present invention may be provided in a folded state, wherein the folded state is effected by folding the crotch part (38) along a folding line (FL) parallel to the longitudinal axis LX. The folded state may be effected by usage of the primary and/or secondary fastening means provided on the crotch part (38) discussed in further detail below, or may be effected by other bonding methods such as ultrasonic energy, heat welding, pressure, or combinations thereof. Whatever the bonding method, and wherever the means for providing the folded state, such measures for providing the folded state is considered a Folded Attachment Means. The Folded Attachment Means may have a peel strength of from about 0.3N to about 10N, or from about 1N to about 5N, according to the Peeling Strength Measurement method herein.

### Belt Part

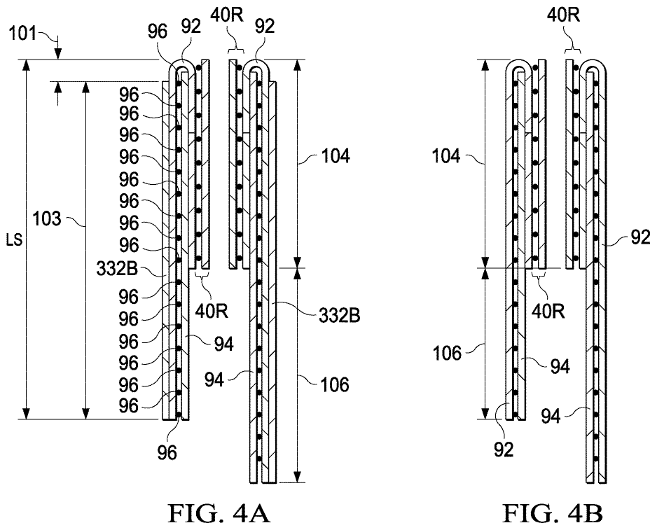

Referring to FIGS. 2A-3D, the belt part (40) comprises a front belt (84) and a back belt (86) extending in the transverse direction between the side seams (32), forming a ring-like elastic part. Referring to FIGS. 4A-4B, the belt part (40) may be made by 2 or more layers of nonwoven substrates (92, 94) with elastic bodies sandwiched between the nonwoven layers. The front and back belts (84, 86) may be imparted elasticity by elastic bodies, for example elastic strands (96) laminated between nonwoven substrates. The elastic bodies may be strips of elastic films, or elastic strands (96) having a density of from about 400 dtex to about 1200 dtex which are visible to the naked eye. The elastic bodies may be low dtex elastic strands which are less discernible to the naked eye and thus provide an undergarment appearance to the belt part (40), such as those having a density of from about 30 dtex to about 400 dtex, or from about 30 dtex to about 300 dtex. In discussions hereunder, the front belt (84) and the back belt (86) may each be expressed in zones wherein multiple zones span in the transverse direction and defined by its location from the distal edge to the proximal edge relative to the percentage of the seam length LS wherein the distal edge is considered 0% and the proximal edge is considered 100%.

Referring to FIGS. 2A-3D, and 14A, the belt part (40) of the present invention comprising a front and back belt (84, 86) may comprise a concave belt notch (108) on at least one of the front belt (84) or the back belt (86), wherein the belt notch (108) positioned on the proximal edge and having a longitudinal dimension of from about 20% to about 40% of the respective belt, the belt notch (108) positioned on the transverse center and having a transverse dimension of from about 10% to about 30% of the respective belt. The longitudinal and transverse dimensions of the belt notch (108) is measured in the contracted state of the belt part (40). Further, the remainder of the proximal edge of the belt comprising the belt notch (108) is a side proximal edge (90S) wherein the side proximal edge (90) is parallel with the distal edge, wherein the angle between the side proximal edge and the belt notch (108) may be from about 80 degrees to about 120 degrees, or from about 90 degrees to about 120 degrees in the contracted state according to the Angle of Belt notch measurement method herein. The angle between the side proximal edge and the belt notch may be from about 80 degrees to about 120 degrees, or from about 90 degrees to about 120 degrees in the extended state as well, according to the Angle of Belt notch measurement method herein. Without being bound by theory, by providing a belt notch (108) in such configuration, the belt part (40), when formed with the crotch part (38), is prevented from being soiled by bodily exudates, while also providing better coverage. Better coverage is effected by preventing making a gap between the belt part (40) and crotch part (38) by having an appropriate amount of force maintained along the proximal edge (90). Further, such belt notch (108) enables the crotch part (38) to face the exudate point and vicinity of the wearer, thus provides good absorbency and prevents leakage of the assembled absorbent article (20).

Referring to FIGS. 2A-2B, 3A-3B and 4A-4B, the front belt and the back belt (84, 86) may each comprise: an anchoring zone (104) comprising a reinforcement material (40R) positioned at from about 0% to about 70%, or from about 0% to about 60% of the seam length LS. At least a portion of the anchoring zone (104) may superpose the primary belt fastening means (331B). The reinforcement material (40R) may be on the wearer facing side or the garment facing side of the belt part (40), or the wearer facing side. The reinforcement material (40R) may be a material which does not interfere with the transverse stretchability of the belt part (40), such as an additional laminate comprising nonwoven and elastic strands, a stretchable film, or a stretchable nonwoven.

The front and back belts (84, 86) may each comprise; a tummy zone (106) positioned proximal from the anchoring zone (104), and a waist zone (102, not shown) positioned distal from the anchoring zone (104). When the front and back belts (84, 86) have different longitudinal dimensions, the remainder of each of the front belt (84) and the back belt (86) are considered the waist zone (102) and the tummy zone (106). The anchoring zone (104) may be provided to have at least 1.5 times, or from about 2.2 to about 10 times the tensile stress of the tummy zone (106). The anchoring zone (104) may be provided to have at least 2 times, or from about 2.2 to about 5 times, the bending force of the tummy zone (106). The anchoring zone (104) may be provided to have at least 1.5 times, or from about 1.6 to about 4 times, the tensile stress of the waist zone (102). The anchoring zone (104) may be provided to have at least 2 times, or from about 2 to about 5 times, the bending force of the waist zone (102). By providing the anchoring zone (104) to have higher tensile stress and/or higher bending force compared to the remaining zones, this provides better fit for the assembled pant absorbent article. The anchoring zone (104) of the front belt (84) and the back belt (86) may be so arranged such that the position relative to 0% to 100% of LS is matched between the front belt and the back belt (84, 86) for good appearance.

Referring to FIGS. 2A-2B and 3A-3B, the tensile stress of the fastening zone (103) may not be higher than the tensile stress of the remaining zones. The front and back belts (84, 86) may each comprise; an upper zone (101) positioned distal from the fastening zone (103), and a lower zone (105, not shown) positioned proximal from the fastening zone (103). When the front and back belts (84, 86) have different longitudinal dimensions, the remainder of each of the front belt (84) and the back belt (86) are considered the upper zone (101) and the lower zone (105). When the front belt and back belt (84, 86) each comprise an upper zone (101) distal from the fastening zone and a lower zone (105) proximal from the belt fastening zone, the upper zone (101), the fastening zone (103), and the lower zone (105) may have substantially the same tensile stress. By substantially the same tensile stress, what is meant is that among the 2 or 3 values obtained, any value is within ±10% of the other value, or the median value. The upper zone (101) may have higher tensile stress than the fastening zone (103).

The belt part (40) may be made of durable material including woven or knitted textiles made of natural fibers such as cotton, linen, wool, bamboo, hemp, silk, rayon, and the like, as well as blends of any of these fibers with any other(s), or with synthetic fibers. Examples of synthetic fibers suitable for use as components of durable materials include polyester, nylon, spandex and/or other elastomer fibers. Durable materials also may include breathable water repellent materials such as GORE-TEX (a product of W. L. Gore & Associates, Inc., Elkton, MD), fabrics comprising microencapsulated phase-change polymer materials such as OUTLAST COMFORTEMP fabrics (products of Outlast Technologies, Boulder, CO as in U.S. Pat. Nos. 6,514,362 and 6,207,738), COOLMAX (a product of Invista, Wichita, KS), and the like. Suitable durable material may be formed in any weave or knit fabric form, including birdseye fabric, terry, fleece, flannel, knits, stretch knits, sherpa, suedecloth, microfleece, satin, velour, Burley knits, etc. Suitable examples include POLARTECH POWER DRY, POWER STRETCH and WIND PRO (products of Polartec, LLC, Lawrence, MA). Knitted textiles may impart good fit, comfort and/or appearance to the belt part (40). Incorporation of fibers of spandex or other elastomer may also enhance stretchability and elasticity, and thereby impart good fit, comfort and/or appearance to the belt part (40), than textiles not including such elastomeric fibers. Specific suitable examples for durable materials include jersey knits of blends of rayon (93%) and spandex (7%) fibers; modal (94%) and spandex (6%) fibers; cotton and spandex fibers; and bamboo and spandex fibers.

### Crotch Part

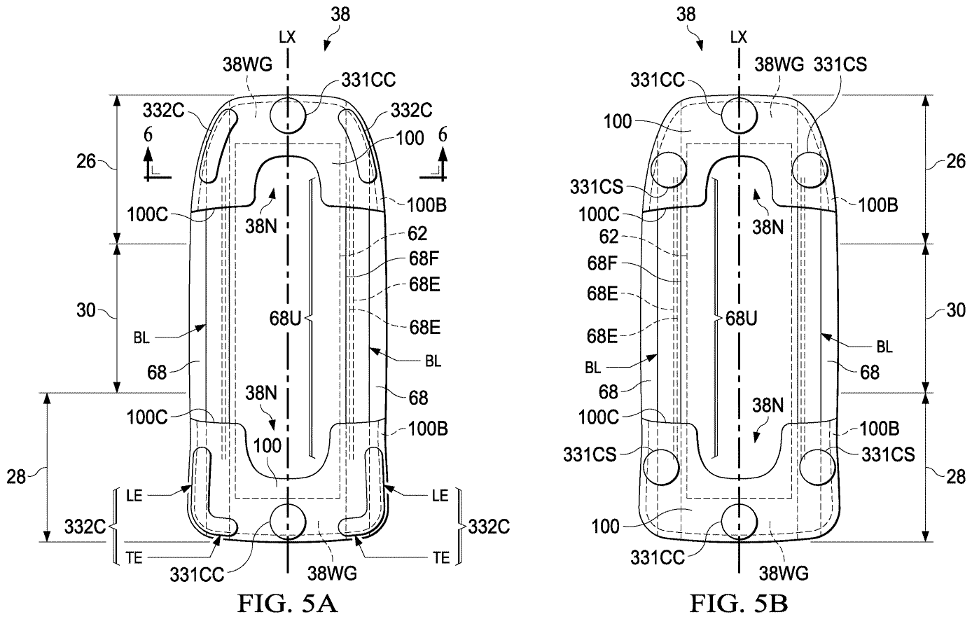

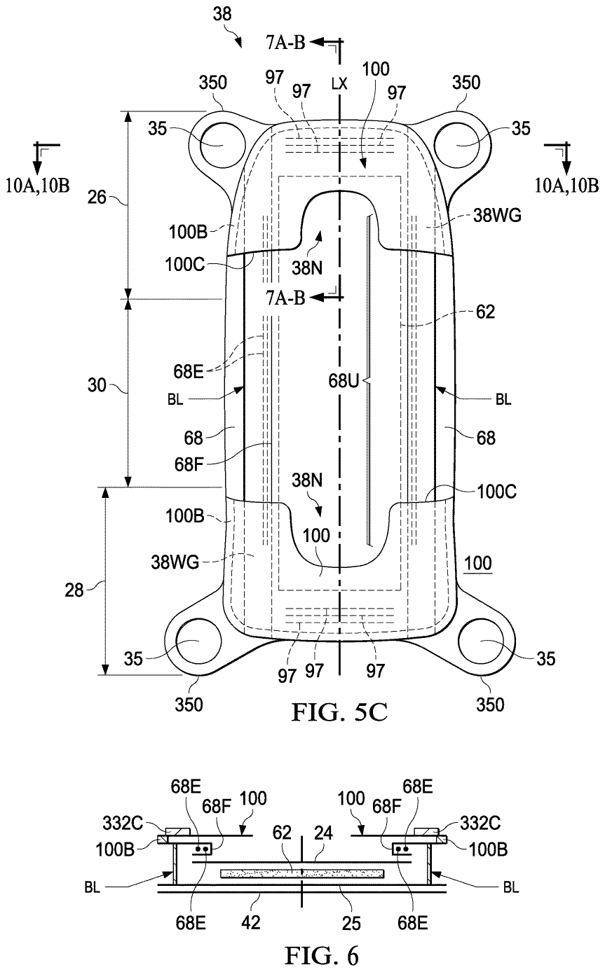

Referring to FIG. 5A-5C, the crotch part (38) comprises a front region (26), a center region (30), and a back region (28) each extending one third of the longitudinal dimension of the crotch part (38). Referring to FIG. 6, the crotch part (38) comprises a water permeable topsheet (24), a water impermeable backsheet (25), an absorbent core (62) disposed between the topsheet (24) and the backsheet (25). The crotch part (38) may further comprise an outer cover layer (42).

The water permeable topsheet (24) is positioned at least in partial contact or close proximity to a wearer. Suitable topsheets (24) may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet (24) is generally supple, soft feeling, and non-irritating to a wearer's skin. The topsheet (24) is liquid permeable, permitting bodily fluids to readily penetrate through the thickness of the topsheet (24). Exemplary topsheets (24) suitable herein include those available from Xiamen Yanjan New Material Co. Ltd made of carded nonwoven substrate comprising PE/PET bi-component fibers, Fibertex NiLai, Malaysia with tradename H30501221 or FQN Hazlet NJ with tradename SB1206169. Any portion of the topsheet (24) may be coated with a lotion or skin care composition as is known in the art. Examples of suitable lotions include those described in U.S. Pat. Nos. 5,607,760; 5,609,587; 5,635,191; and 5,643,588.

The water impermeable backsheet (25) is designed to prevent the exudates absorbed by and contained within the absorbent core (62) from soiling articles that may contact the pant diaper article (20), such as bed sheets and undergarments. The backsheets (25) may be positioned such that it extends beyond the absorbent core (62) disposed on the wearer-facing side of the particular backsheet (25) in both the longitudinal direction and the transverse direction. Suitable backsheet (25) materials include films such as those manufactured by Plaster Argentina with tradename PLBA NBBS 10-12GSM PR V1. Other suitable backsheet (25) materials may include breathable materials that permit vapors to escape from the pant diaper article (20) while still preventing exudates from passing through the backsheet (25). Exemplary breathable materials may include materials such as woven webs, nonwoven webs, composite materials such as film-coated nonwoven webs, and microporous films such as manufactured by Daika Japan with tradename MPF DKH-180 15G V7 and manufactured by Berry Nashville, TN with trademark BR-137P V13. Such breathable composite materials are described in greater detail in PCT Application No. WO 95/16746 and U.S. Pat. No. 5,865,823. Other breathable backsheets including nonwoven webs and apertured formed films are described in U.S. Pat. No. 5,571,096. An exemplary, suitable backsheet is disclosed in U.S. Pat. No. 6,107,537. Other suitable materials and/or manufacturing techniques may be used to provide a suitable backsheet including, but not limited to, surface treatments, particular film selections and processing, particular filament selections and processing, etc.

The crotch part (38) of the present invention comprises an absorbent core (62) for absorbing and containing bodily exudates disposed on the wearer facing side. The absorbent core (62) may include an absorbent layer and an acquisition system. The absorbent layer is the region wherein absorbent materials having a high retention capacity, such as superabsorbent polymers, are present. The absorbent layer may be substantially cellulose free. Alternatively, the absorbent layer may contain cellulose. There may be an absorbent layer mainly comprising cellulose, and another absorbent layer mainly comprising superabsorbent polymers.

Superabsorbent polymers of the absorbent layer may be disposed between first and second layers of material immobilized by a fibrous layer of thermoplastic adhesive material. The first and second layers of materials may be nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. Some portions of the absorbent layers may be configured to have substantially no absorbent material to form a channel or a plurality of channels. Channels may be useful for allowing the absorbent core (62) to bend upon swelling with fluids, such that the center region (30) conforms to the wearer's body after swelling and prevent sagging of the diaper article (20). The absorbent layers may be disposed in plurality in the absorbent core (62).

Alternatively, the absorbent core (62) may comprise a high loft material encompassing superabsorbent polymers. The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft material (without the superabsorbent particles) may have a density at a pressure of 4.14 kPa (0.6 psi) below 0.20 g/cm³, in particular ranging from 0.05 g/cm³ to 0.15 g/cm³. The high loft layer (without the superabsorbent particles) may have a density at a pressure of 2.07 kPa (0.3 psi) below 0.20 g/cm³, in particular ranging from 0.02 g/cm³ to 0.15 g/cm³. The high loft layer (without the superabsorbent particles) may have a density at a pressure of 0.83 kPa (0.12 psi) below 0.15 g/cm³, in particular ranging from 0.01 g/cm³ to 0.15 g/cm³, and a basis weight of from 15 to 500 gsm, preferably 30~200 gsm, such as those described in US 2021/0361497 A1. The absorbent core (62) comprising high loft material encompassing superabsorbent polymers may also contain channels.

The absorbent core (62) may also contain an acquisition system for facilitating the acquisition and the distribution of body exudates, and may be placed between the topsheet (24) and the absorbent layer. The function of the acquisition system is to rapidly acquire the fluid from the topsheet (24) away from the wearer-facing side and/or to distribute over a larger area so it is more efficiently absorbed by the absorbent core. The acquisition system may include cellulosic fibers. Channels may also be formed in the acquisition system, and may be configured to at least partly match the channels of the absorbent layer in the thickness direction. It is also possible that such another liquid management layer may be placed between the backsheet (25) and the absorbent layer. The liquid management layer may be a spunlace nonwoven comprising viscose, PET, CoPET/PET fibers, and combinations thereof.

Referring to FIGS. 5A-5C, the crotch part (38) may comprise an inner pocket (38WG) along at least one of the front or back ends, wherein the inner pocket (38WG) is formed by a pocket substrate (100) which is hydrophobic and comprising a concave pocket notch (38N) in the transverse center, wherein the pocket substrate (100) is disposed along the front and/or back edges of the crotch part (38) and partially bonded by a bonding region (100B) to the remainder of the crotch part (38) in order to form the inner pocket (38WG) open towards the longitudinal center of the crotch part (38). Such bonding between the pocket substrate (100) and the remainder of the crotch part may be provided by ultrasonic energy. The opening of the inner pocket (38WG) towards the longitudinal center forms a proximal side contour (100C), which proximal side contour includes the shape of the concave pocket notch (38N) in the transverse center. The pocket substrate (100) may be disposed along both the front end or the back ends of the crotch part (38) as providing a base for bonding, for example, a discrete material carrying hooks of a hook-and-loop fastening system, bonded on the wearer-facing side of the pocket substrate (100), as discussed in further detail below.

The crotch part (38) may comprise a pair of inner cuffs (68) disposed along both transverse edges extending in the longitudinal direction and superposing the topsheet (24). The inner cuffs (68) may be made of material having high fluid impermeability to prevent leakage of bodily exudates. Each inner cuff (68) extends from the backsheet (25) toward the longitudinal axis to form a free edge (68F). The inner cuffs (68) may be made by providing a part comprising a cuff material having high fluid impermeability with elastic members (68E) provided in attachment, followed by bonding the cuff material along the left and right side edges of the backsheet (25), wherein the longitudinally extending bonding line defines a base (BL) of the inner cuff. The elastic member (68E) may be disposed adjacent the free edge (68F) for providing elasticity to the inner cuff (68), for example from about 0.5 mm to about 3 mm from the free edge (68F). The elastic member (68E) may be provided in plurality of strands with an interval of from about 1 mm to about 2 mm. Each inner cuff (68) of the present invention comprises an active portion (68U) unattached to the topsheet (24) and extending from the front region (26) through the crotch region (30) to the back region (28) which enables a longitudinally extending inner cuff elastic member (68E) in active elasticity such that the inner cuffs (68) are kept in close proximity with the wearer to provide a barrier effect. The elastic strand (68E) existing closest to the free edge (68F) may have the same or lower tensile stress than the remaining elastic strands. The elastic strands (68E) toward the front and back ends may be deactivated. In FIG. 4, only the inner cuff elastic strands (68E) in active elasticity are depicted.

The inner cuffs (68) and the pocket substrates (100) forming the inner pocket (38WG) may be left unbonded. The active portion (68U) of the inner cuff elastics may be superposing the proximal side contour (100C) of the inner pocket. Without being bound by theory, by providing the active portion (68U) of the inner cuff elastics and proximal side contour (100C) in this relationship, the inner pocket (38WG) may be opened in the front and/or back regions where it exists due to the force provided by the inner cuff elastic members (68E). Further, by providing the inner cuffs (68) and pocket substrate (100) forming the inner pocket (38WG) unbonded, this maintains flexibility in this region.

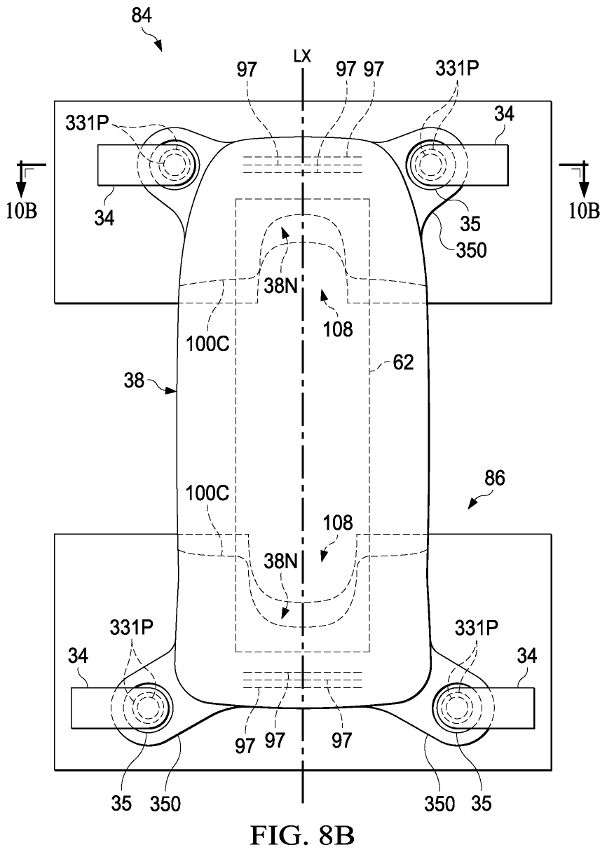

Referring to FIGS. 5A-5C, the pocket substrate (100) may extend to the proximal side such that the inner pocket (38WG) may superpose the absorbent core for improved containment. Referring to FIGS. 8A-8B, the belt part (40) may also be provided with a belt notch (108). Such belt notch (108) may be provided on the belt part (40) that is planned to be attached with the crotch fastening means (33C) having the inner pocket (38WG). By the combination of the belt notch (108) and the pocket notch (38N) of the inner pocket (38WG), this may enable space for the crotch pocket substrate to lift from the topsheet (24) and thus provide good containment. When the belt part (40) and the crotch part (38) are assembled in the intended position, any part of the inner pocket (38WG) of the crotch part may be so configured to exist distal from the concave notch (108) of the front belt or the back belt.

Referring to FIG. 6, the crotch part (38) may further comprise an outer cover (42) disposed on the garment facing side of the backsheet (25) for providing the diaper article in a finished appearance. The outer cover (42) may be made of a soft, non-woven material. Exemplary outer cover (42) suitable herein include those available from Guanhe Hygiene Products Co., Ltd with tradename S31921A made of spunbond nonwoven substrate comprising PP fiber, and Fibertex NiLai Malaysia with tradename A10160EJ-MALAYSIA and available from FQN Hazlet NJ with tradename SM1104174.

### Primary Fastening Means

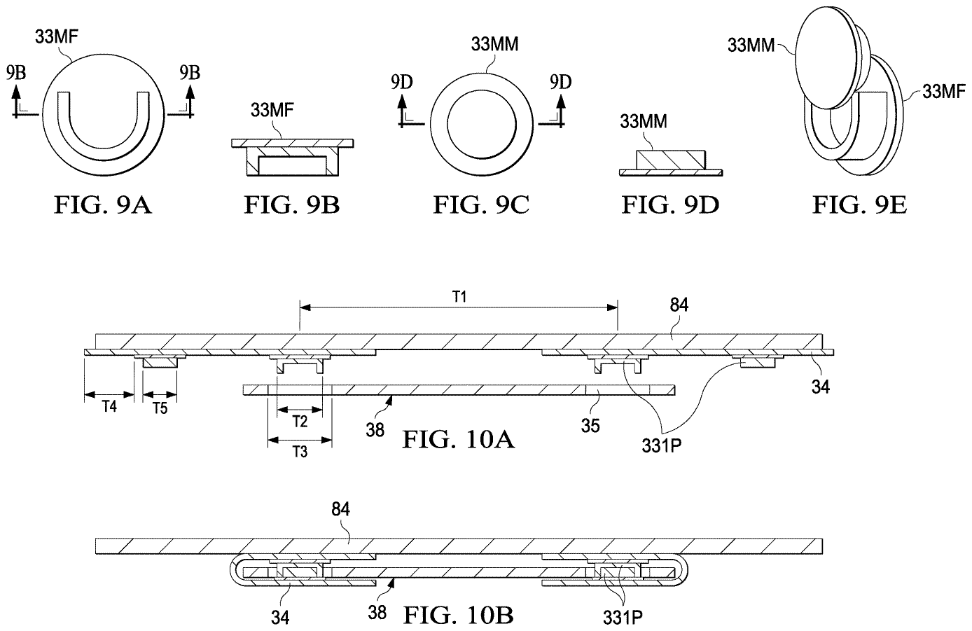

The belt part (40) and the crotch part (38) of the present invention are detachable with each other by a primary belt fastening means (331B) and a primary crotch fastening means (331C) fastened magnetically. The primary fastening means (331) may be provided by a pair of magnet pieces of opposite polarity, suitably a pair of magnet pieces of opposite polarity having a locking means for maintaining the pair of magnet pieces of opposite polarity fastened. The locking means may be a simple physical lock, for example a slit, a rotating means, or combination thereof. For example, referring to FIGS. 9A-9E, the pair of magnet pieces may comprise a protruding male portion (33MM) and a receiving female portion (33MF), wherein the slit is provided on the female portion (33MF) for accepting the male portion (33MM) according to gravity. By providing the slit in such configuration, reversing the acceptance of the male portion (33MM) against gravity is difficult, thus this avoids the pair of magnets being accidentally unengaged due to the movement of the wearer.

Referring to FIGS. 2A-2B, 3A-3B, 5A-5B, and 8A, the primary belt fastening means (331B) may be a magnet having a polarity, and the primary crotch fastening means (331C) may be a magnet of opposite polarity of the primary belt fastening means (331B). The primary belt fastening means (331B) may be provided on the garment facing side of both the front belt and the back belt (84, 86), and the primary crotch fastening means (331C) may be provided on the wearer-facing side on corresponding positions of the front and back sides. Accordingly, referring to FIGS. 1A-1D, the primary belt fastening means (331B) may serve as the fastening belt indicia, and the primary crotch fastening means (331C) may serve as the fastening crotch indicia. Referring to FIGS. 2A-2B, 3A-3B, 5A-5B and 8A, the primary belt fastening means (331B) may be provided in one or a plurality of magnet pieces, the primary crotch fastening means (331C) may be provided in the same number of magnet pieces as the primary belt fastening means (331B). The primary belt fastening means (331B) may comprise a center belt magnet piece (331BC) positioned on the longitudinal centerline of the pant absorbent article, and the primary crotch fastening means (331C) may comprise a center crotch magnet piece (331CC) positioned on the longitudinal axis of the pant absorbent article. By providing the primary fastening means (331) on the longitudinal axis of the resulting pant absorbent article, this provides easy application and assembling of the belt part (40) and crotch part (38) in the intended position. Referring to FIGS. 2B, 3B, and 5B, the primary crotch fastening means (331C) may further comprise a pair of side crotch magnet pieces (331CS) positioned on the left and right side edges of the crotch part (38), and the primary belt fastening means (331B) may further comprise a pair of side belt magnet pieces (331BS) in the corresponding position of the side crotch magnet pieces (331CS). By providing additional primary fastening means on the left and right side edges of the crotch part (38), this provides further secured fastening. Corresponding to these belt part configurations of FIGS. 2A-2B and 3A-3B, the front region (26) of the crotch part (38) may be tapered such that the longitudinal edge of the front region has the smallest transverse dimension in the front region (26) as in FIGS. 5A-5B. Such overall contoured shape enables smooth and secure attachment of the crotch part (38) on the front side to the belt part (40) on the front side, and further provides sustained fit during wear.

Referring to FIG. 5B, when the primary crotch fastening means (331C) comprises a pair of side crotch magnet pieces (331CS) positioned on the left and right side edges of the crotch part (38), the left and right magnets may be provided in opposite polarity in order to suitably serve as a Folded Attachment Means utilizing the primary crotch fastening means (331C). The side crotch magnet pieces (331CS) on the front and back regions on the left side may have one polarity, and the side crotch magnet pieces (331CS) on the front and back regions on the right side may have the opposite polarity. Alternatively, when there are a plurality of magnet pieces on the crotch part (38), these magnet pieces may all have the same polarity. By providing magnet pieces on the crotch part (38) to all have the same polarity, this avoids magnets on the crotch part (38) to unintentionally stick to each other upon assembling the absorbent article, or otherwise.

Referring to FIGS. 2C-2D, 3C-3D, 5C, and 8B, the primary belt fastening means (331B) may comprise a combination of opposite magnets (331P) having opposite polarity, and the primary crotch fastening means (331C) may comprise an engaging means with the primary belt fastening means (331B). What is meant by “combination of opposite magnets” or “opposite magnet combination” herein is a pair of magnets having opposite polarity both disposed on the belt part (40), wherein the primary crotch fastening means (331C) comprises an engaging means to engage with such opposite magnet combination. Such configuration is advantageous in providing the magnet pieces only on the belt part (40), which belt part (40) is intended to be used more than once. Accordingly, this enables avoiding any magnet pieces to be on the crotch part (38), thus conserving material. Referring to FIGS. 2C-2D, 3C-3D, 8B, and 10A-10B, the primary belt fastening means (331B) may further comprise a strap (34) which enables engaging with the engaging means for effecting the combination of opposite magnets (331P) to attach to each other. The strap (34) may be made of substantially non-elastic material, and the opposite magnet combination (331P) may be bonded to the strap (34), and at least a portion of the strap (34) may be bonded to the belt part (40). The substantially non-elastic material for making the strap (34) may be selected from the group consisting of cotton, linen, wool, bamboo, hemp, silk, rayon, polyester, nylon, and combinations thereof. By providing the strap (34) substantially non-elastic, the primary fastening means (331) is stably maintained in the same position even after loading of the crotch part (38).

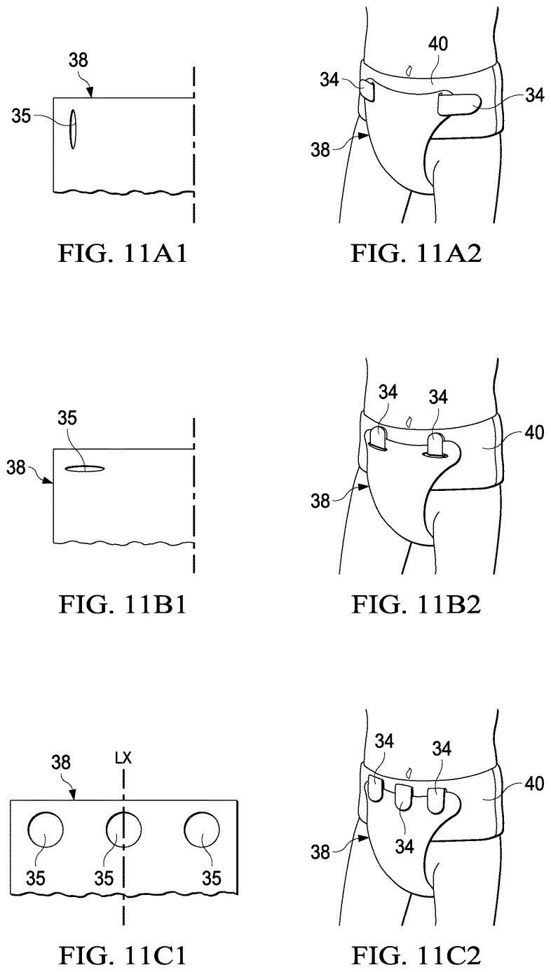

The engaging means on the crotch part (38) may be an opening (35) through which the strap (34) is movable. For example, referring to FIGS. 5C, 10A-10B and 11C1-11C2, the opening (35) engaging means may be a circular opening (35) similar to the shape of the magnet used as the primary belt fastening means (331B). Referring to FIGS. 11A1-11A2 and 11B1-11B2, the opening (35) may be a slit for moving the strap (34) through.

Referring to FIGS. 10A-10B, when each magnet piece of the opposite magnet combination (331P) is substantially a circle and the magnet piece having a greater diameter has a diameter of T2, the opening (35) to engage with the opposite magnet combination (331P) may be substantially a circle having a diameter of T3, wherein the following relationship is met: T2<T3<T2×1.5. By providing dimensions T2 and T3 in such relationship, this enables easy engaging of the opposite magnet combination (331P). Each magnet piece of the opposite magnet combination (331P) may be provided at least about 5 mm away from, or from about 10 mm to about 20 mm away from the edge of the strap (34). By providing the magnet pieces of the opposite magnet combination (331P) away from the very edge, this enables easy manipulation of the opposite magnet combination (331P).

Referring to FIGS. 5C and 8B, when the primary belt fastening means is provided in opposite magnet combination (331P), the primary crotch fastening means (331C) comprises an engaging means with the primary belt fastening means (331B). The engaging means may be an opening (35) positioned on the left and right side edges of the crotch part (38), wherein a pair of side belt opposite magnet combinations (331PS) may be provided on the belt part (40) in the corresponding position. By providing the opening (35) on the left and right side edges of the crotch part (38), this provides secure fastening, and good appearance after fastening. The overall shape of the crotch part (38) may be provided such that the minimum transverse dimension of the center region (30) is smaller than those of the front region (26) and the back region (28), the longitudinal edge of the front region (26) is concave, and the longitudinal edge of the back region (28) is concave. Such overall contoured shape enables smooth inserting of the crotch part (38), in the folded form, between the legs of the wearer upon assembling the pant absorbent article. The crotch part (38) may be provided with an additional protruding material (350) for providing the opening (35) and forming the aforementioned concave shape along the longitudinal edges of the front and back regions (26, 28). The additional protruding material (350) may be sandwiched between layers forming the crotch part (38) and extending therefrom, or attached to the garment facing side of the crotch part (38). Alternatively, the overall shape of the crotch part (38) may be substantially rectangular, which is economical to form (not shown).

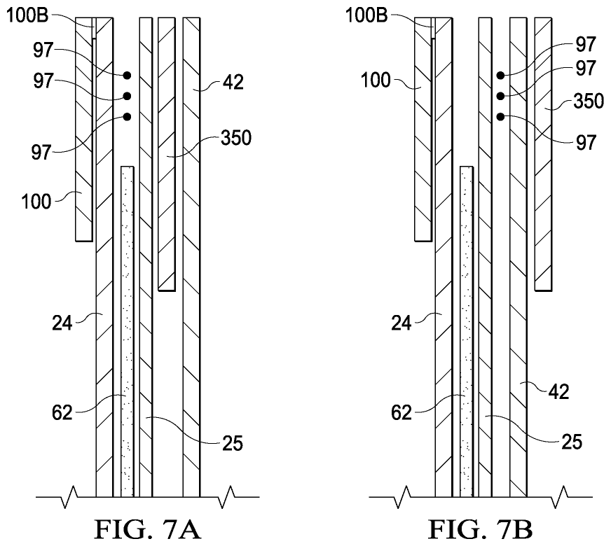

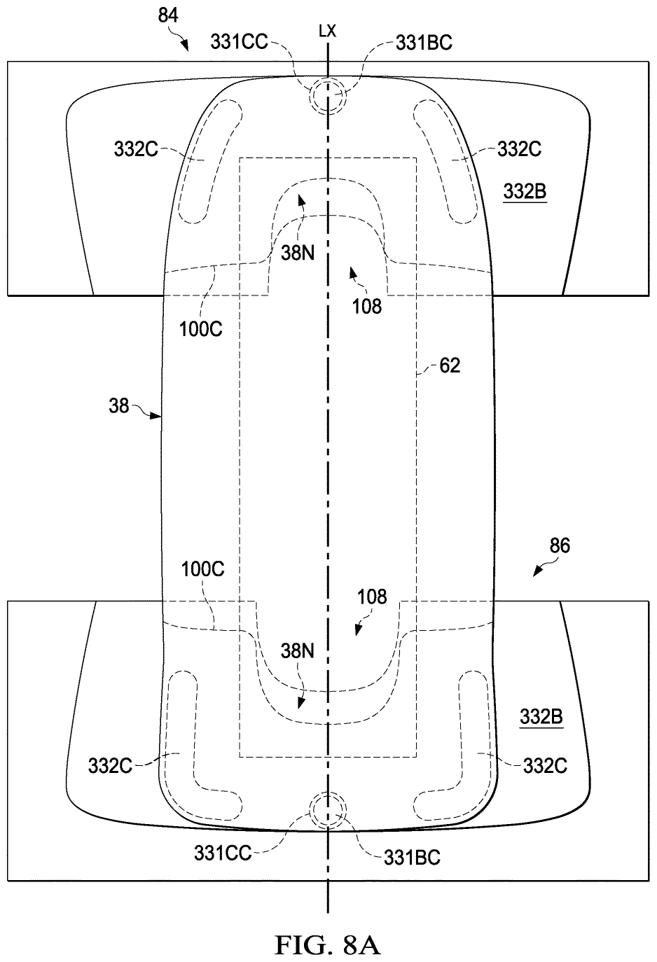

Referring to FIGS. 5C and 7A-7B, the crotch part (38) of this configuration may comprise an edge elastic element (97) positioned adjacent the front and/or back edge of the crotch part (38) and extending in the transverse direction. The edge elastic element (97) may secure close fit of the transverse center of the front and back edges of the crotch part (38) against the belt part (40). Referring to FIG. 7A, the edge elastic element (97) may be disposed between the topsheet (24) and the backsheet (25), while the additional protruding material (350) may be disposed between the backsheet (25) and the outer cover layer (42). Referring to FIG. 7B, the edge elastic element (97) may be disposed between the backsheet (25) and the outer cover layer (42), while the additional protruding material (350) may be disposed on the garment facing side of the outer cover layer (42).

Referring to FIGS. 10A-10B, when the belt part (40) is provided with a pair of side belt opposite magnet combinations (331PS), the magnet pieces from the left and right side belt opposite magnet combinations which are positioned closest to each other may be positioned at a distance of T1. Distance T1 is measured from the center of each magnet piece. T1 may be at least about 50 mm, or from about 70 mm to about 150 mm. By providing distance T1 in such dimension, this enables easy fastening of the primary fastening means (331).

Referring to FIG. 11C1-11C2, the primary belt fastening means (331B) may comprise a center belt opposite magnet combination (331PC) positioned on the longitudinal centerline of the pant absorbent article, and the primary crotch fastening means (331C) may comprise a center crotch opening (35) positioned on the longitudinal centerline of the pant absorbent article.

### Secondary Fastening Means

The pant absorbent article of the present invention may comprise a secondary fastening system (332) in addition to the primary fastening system (331). Referring to FIGS. 2A, 3A, and 5A and 8A, the secondary fastening system (332) may be a hook and loop fastening system, wherein the secondary crotch fastening means (332C) comprises hooks, and the secondary belt fastening means (332B) comprises loops. The secondary fastening system may be a pressure sensitive adhesive agent only provided on the crotch part (38). The secondary fastening system (332) assists or cooperates with the fastening provided by the primary fastening system (331) for providing a secure fastening for the assembled pant absorbent article (20).

The secondary belt fastening means (332B) may be loops on the garment facing side of the front and back belts (84, 86) and the secondary crotch fastening means (332C) may be on the wearer-facing side in the front region (26) and the back region (28) matching the positions of the secondary belt fastening means (332B). Referring to FIG. 5A, the crotch part (38) may comprise a pair of side hook regions comprising secondary belt fastening means (332B) positioned on the left and right side edges of the crotch part (38). Such secondary fastening means (332) may be suitably combined with a primary fastening means (331) on the longitudinal axis. By the combination of the primary fastening means (331) on the longitudinal axis and the secondary fastening means (332) on the left and right side edges of the crotch part, this may provide a secure fastening means for the resulting pant absorbent article. While not shown, the crotch part (38) may comprise a secondary crotch fastening means (332C) positioned on the longitudinal axis, and a primary crotch fastening means (331C) may be provided on the left and right side edges of the crotch part (38). By the combination of the primary fastening means (331) on the left and right side edges of the crotch part and the secondary fastening means (332) on the longitudinal axis, this may also provide a secure fastening means for the resulting pant absorbent article.

Referring to FIG. 5A, the hooks fastening means on the crotch part (332C) may be provided on the wearer-facing side of the pocket substrate (100) for forming the inner pocket (38WG), as explained above. The area of the hooks fastening means on the crotch part (332C) may or may not match the area of the pocket substrate (100). The hooks fastening means on the crotch part (332C) may only be disposed adjacent the front and back longitudinal edges. Thus, the hooks may not overlap the absorbent core (62). Each hooks fastening means on the crotch part (332C) may comprise a transverse element (TE) extending in the transverse direction adjacent the longitudinal end of the crotch part (38) and a longitudinal element (LE) extending in the longitudinal direction adjacent the transverse edges of the crotch part (38). Providing the hooks crotch fastening means on the crotch part (332C) in this region is beneficial in that it provides good finished appearance, and also provides good prevention of leakage between the belt part (40) and the crotch part (38). The longitudinal element (LE) of the hooks fastening means on the crotch part (332C) may be continuous with the transverse element (TE) for securing good fastening. The longitudinal element (LE) may be positioned away from the longitudinal edge of the crotch part (38), and transverse element (TE) may be positioned away from the transverse edge of the crotch part (38) for material saving.

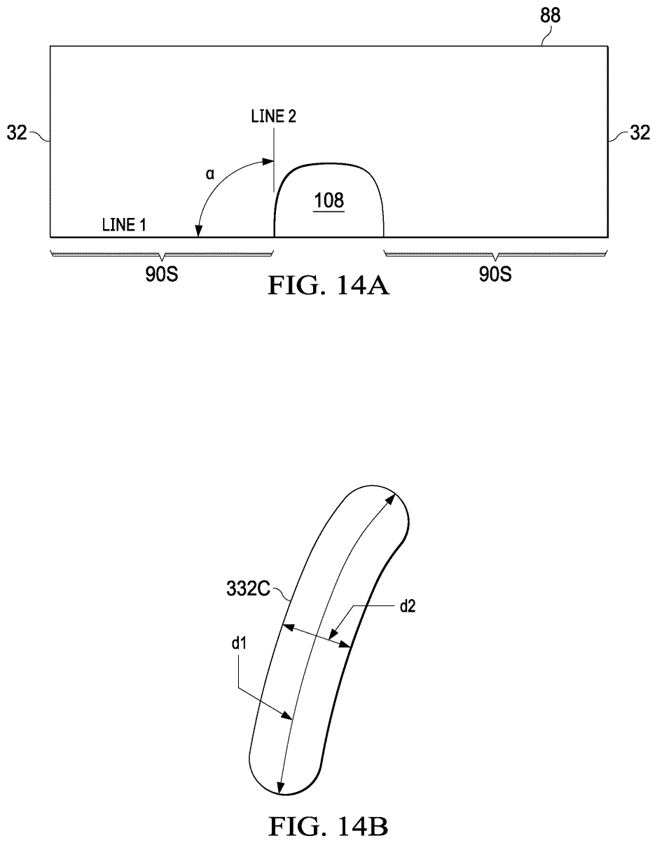

Referring to FIG. 14B, the hooks fastening means on the crotch part (332C) may have a greater dimension (d1) along the curvature of the front or back end of the crotch part (38) and a smaller dimension (d2) perpendicular to the greater dimension (d1). The greater dimension (d1) of the hooks fastening means is measured along the center line of the fastening means. The smaller dimension (d2) of the hooks fastening means is taken at the middle point of the greater dimension (d1).

In yet another embodiment, a magnet forming the primary crotch fastening means (331C) may be bonded on a material detachable to the secondary crotch fastening means (332C), for example the magnet may be bonded on a loops material of a hook-and-look fastening system, which loops material is detachable to the hooks crotch fastening means (332C) on the crotch part. By providing the primary crotch fastening means (331C) detachable from the crotch part (38), the primary crotch fastening means (331C) may be re-used on another crotch part (38) and conserve material.

The secondary crotch fastening means (332C) provided as hooks may suitably serve as the Folded Attachment Means for the crotch part (38). The Folded Attachment Means may have a peel strength of from about 0.3N to about 10N, or from about 1N to about 5N, according to the Peeling Strength Measurement method herein.

### Peeling Strength Measurement

### 1. Preparation of Finished Product Specimen

Specimen for the measurements hereinbelow are obtained from 3 finished parts.

For measuring the crotch part (38), the crotch part (38) in folded state with hooks or bondings inside the folded crotch part (38) is used as is. To obtain test specimens, the longitudinal direction of the crotch part (38) is identified, and the crotch part (38) is stretched in the longitudinal direction such that any longitudinally extending elastic bodies, such as cuff elastics, are fully stretched, and then the crotch part (38) is attached on an appropriate board having at least an area of the fully stretched article via any attachment means, such as tape, adhesive, hooks, or the like. Identify the front and/or back side attached by Folded Attachment Means. Cut by scissors along the transverse direction at 10 cm from the longitudinal edge of the side having Folded Attachment Means. The portion exposed by such cutting is the measurement edge. Remove the obtained portion from the attachment board, while maintaining the Folded Attachment Means.

For measuring the belt part (40), the belt part (40) in folded state with hooks is used as is. (Belt parts which do not comprise Folded Attachment Means are not subject to this test.) To obtain test specimens, the belt part (40) is cut open along the side seams (32) to obtain the front and back belts. Cold Spray may be used, paying attention not to make wrinkles in the belt part (40), and not to spray on any belt elastic member. If the Folded Attachment Means exists only on either front or back belt, the corresponding belt part is to be tested. If the Folded Attachment Means exists on both front and back belt, both parts are to be tested. Measure 10 mm from where Folded Attachment Means ends towards side seam in transverse direction, and cut by scissors along the longitudinal direction. The portion with Folded Attachment Means exposed by such cutting is the measurement edge.

### 2. MTS Test Setting and Measuring

As equipment, MTS Criterion Model 42 running TW Elite 4.3.1.375 software with standard tensiometer or equivalent is used. All testing is performed in a room maintained at 23±2° C. and 50±5% relative humidity.

The initial distance between the upper grip and lower grip is set to be 10 mm. The specimen is set that the specimen surface is parallel with ground surface, and the upper grip and lower grip each clamps one side of the specimen along transverse edge. A constant rate of extension of 200 mm/min is applied. The elongation measurement is taken from the point where the program starts. The upper grip starts moving up, while the lower grip remains static, resulting in the Folded Attachment Means unfolding. The program stops until the upper grip moves 120 mm. The force (N) is continuously measured by the machine at a sampling rate of 50 Hz.

The Peak force is the maximum force value observed during the measurement process. Measurements from 3 specimens are obtained and averaged up to 0.01N preciseness.

### Whole Article Force Measurement

Force is measured for the belt part (40) or the assembled absorbent article (20) using an Electronic Tensile Tester with a computer interface such as the MTS Criterion C42 running TestWorks 4 Software (available from MTS SYSTEMS (CHINA) CO., LTD) or equivalent instrument. A load cell is selected so that force results for the samples tested will be between 10 and 90% of capacity of the load cell used. The instrument is calibrated according to the manufacturer's instructions. All testing is performed in a room maintained at 23±2° C. and 50±5% relative humidity.

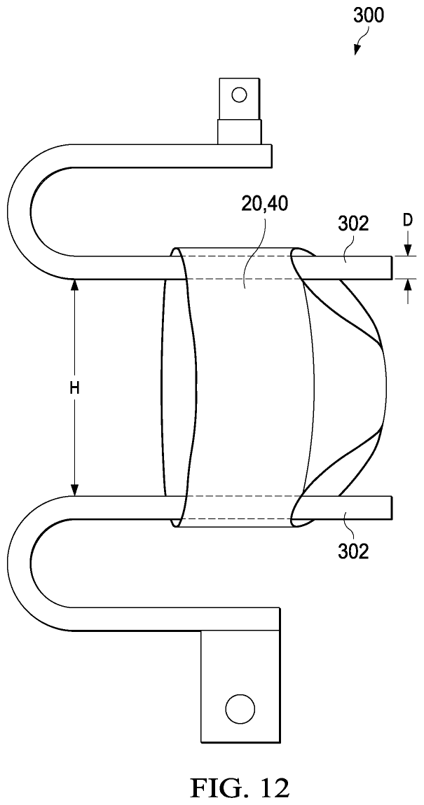

The tensile tester is fitted with hanger-type sample holding fixtures (300) as shown in FIG. 12. Each fixture comprises a rigid linear rubber-coated horizontal bar section (302) to prevent sample slippage during testing. The outer bar diameter (including the rubber coating) of the horizontal bar sections is 10.0 mm. The central axes of the horizontal bar sections (302) are configured to remain parallel and in the same vertical plane throughout the test procedure. The gauge circumference is determined by the following equation:

$$Gauge Circumference = 2 \times ( H + D + π D / 2 )$$

where H is the vertical gap between the horizontal bar sections (302), and D is the outer diameter of the bar.

The instrument is set up to go through the following steps:

| Crosshead Speed | 254.0 | mm/min |
|---|---|---|
| Final Load Point | 19.61 | N |
| Hold Time | 0 | |
| Number of Cycles | 1 | |
| Data Acquisition Rate | 50 | Hz |

A belt part (40) or assembled absorbent article (20) sample is inserted onto the upper horizontal bar section (302) so that the bar passes through the waist opening 35 and one leg opening 35 of the article. The crosshead is raised until the specimen hangs above the lower bar and does not touch lower bar (302). The load cell is tared and the crosshead is lowered to enable the lower bar (302) to be inserted through the waist opening 35 and other leg opening 35 without stretching the article. The article is adjusted so that the longitudinal centerline LX of the article is in a horizontal plane halfway between the upper and lower bars (302). The center of the side portion in contact with the bar (302) is situated on the same vertical axis as the instrument load cell. The crosshead is raised slowly while the article is held in place by hand as necessary until the force is between 0.05 and 0.1N, while taking care not to add any unnecessary force. The gauge circumference at this point is the Initial Gauge Circumference. The test is initiated and the crosshead moves up at 254 mm/min until a force of 19.6N is attained, then the crosshead immediately returns to the Initial Gauge Circumference at the same speed. The maximum circumference at 19.6N and the force at 70% of the maximum circumference during the loading segment and unloading segment of the test are recorded.

The maximum circumference at 19.6N is defined as the Full Stretch Circumference (mm). The Full Stretch Circumference (mm)×0.7 is defined as the 70% Stretch Circumference. The Waist Circumference Force is defined as the force at 70% stretch circumference during the load (extension) segment of the test. Five samples are analyzed and their average are calculated and reported to the nearest 0.01N, respectively.

### Belt Zone Tensile Stress Measurement

This measurement is only provided for belt parts (40) comprising an anchoring zone (104) comprising a reinforcement material (40R). The tensile stress (N/m) is calculated by tensile force (N) divided by the specimen width (m). Force may be measured using an Electronic Tensile Tester with a computer interface such as the MTS Criterion C42 running TestWorks 4 Software (available from MTS SYSTEMS (CHINA) CO., LTD) or equivalent instrument. A load cell is chosen so that force results for the samples tested will be between 10% and 90% of capacity of the load cell. The instrument is calibrated according to the manufacturer's instructions. All testing is performed in a room maintained at 23±2° C. and 50±5% relative humidity. The instrument is equipped with single line contact grips at least as wide as the test specimen.

To obtain test specimens, the sample belt part (40) is cut open along the side seams (32), and to obtain the front and back belts. Cold Spray may be used, paying attention not to make wrinkles in the belt part (40). Care is taken not to spray on any belt elastic member (96). The obtained elastic belt samples are severed into anchoring, waist, and tummy zones (104, 102, 106) according to the present invention with care not to cut any elastic member (96). Samples are pre-conditioned at 23° C.±2 C.° and 50%±5% relative humidity for two hours prior to testing.

The instrument is set up to go through the following steps. Initial Gauge Length is calculated from the Initial Gauge Circumference which is determined during the Whole Article Force Test using separate identical articles, as described above. Initial Gauge Length=0.5×Initial Gauge Circumference. The final gauge length is calculated from the Full Stretch Circumference which is determined during the Whole Article Force Test, as described above.

| | | |
|---|---|---|
| Crosshead Speed | 254.0 | mm/min |
| Data Acquisition Rate | 50 | Hz |

| | |
|---|---|
| Final Gauge Length | 0.5 × Full Stretch Circumference |
| Hold Time | 0 |
| Number of Cycles | 1 |

One end of the specimen is clamped into the upper clamp and the load is tared. The other end of the specimen is clamped into the lower clamp. Approximately 5 mm of each end of the specimen is behind the contact line of the grip. The test is started and the specimen is extended to the final gauge length at a crosshead speed of 254 mm/min, then immediately returned to the original gauge length at the same speed. The specimen is extended in the article transverse direction during the test. The unload force at 70% of the Final Gauge Length during the unload segments of the test is recorded.

Five belt parts are analyzed and the unload forces are recorded for each of the zones. The average tensile force (N) is calculated to the nearest 0.01 N for each. The tensile stress for each zone is calculated by the average tensile force (N) divided by the average specimen width (m) and reported to the nearest 0.1 N/m.

### Bending Force Measurement

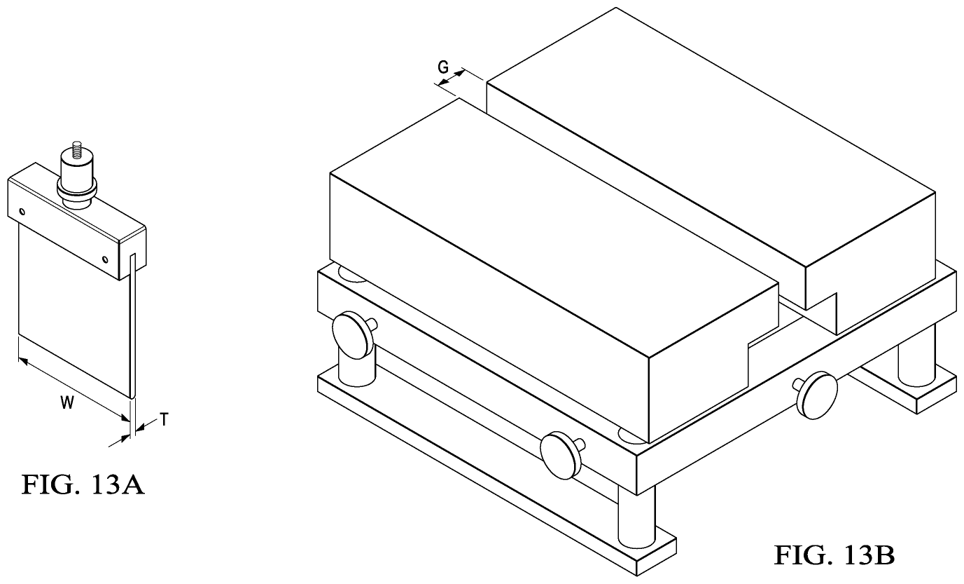

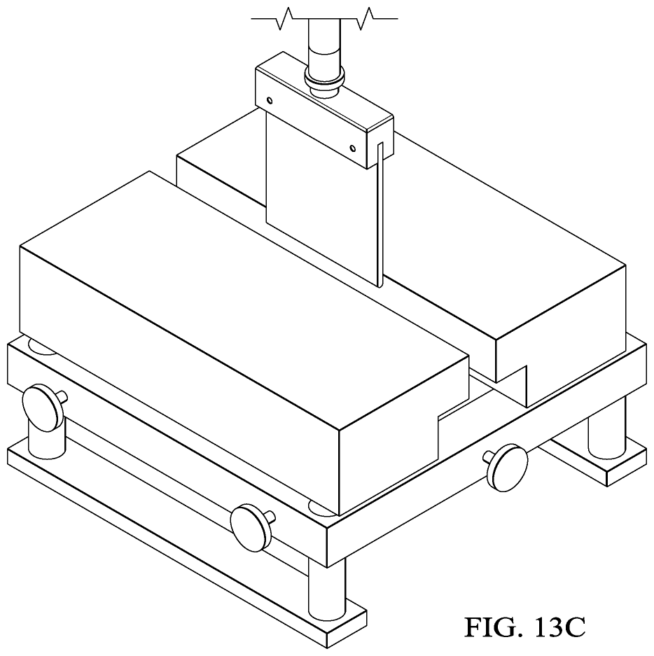

This measurement is only provided for belt parts (40) comprising an anchoring zone (104) comprising a reinforcement material (40R). As equipment, TA (Texture Analyzer) from TAXT plus Stable Microsystems or equivalent instrument is used. The Texture Analyzer is fitted with a 3-point bending blade and bending bridge as shown in FIGS. 13A and 13B. Referring to FIG. 13A, the bending blade has a dimension W of 70 mm, and a dimension T of 3 mm. Referring to FIG. 13B, the dimension G is 10 mm. The instrument is calibrated according to the manufacturer's instructions. All testing is performed in a room maintained at 23±2° C. and 50±5% relative humidity.

To obtain test specimens, the belt part (40) is severed into zones in the same manner as the Belt Zone Tensile Stress Measurement method above.

Initial setting of the instrument is shown in below table. Draw a center line in the length direction on sample. Referring to FIG. 13C, place the prepared sample between the bending blade and bridge, make sure the bending blade on top match the center line. Set the crosshead travel to compress the sample article until travel to the target distance 13 mm, then return to the initial position. Five samples are analyzed. Bending peak load are calculated and reported to the nearest 0.01N.

| Test mode | Compression | |
|---|---|---|
| Test speed | 0.5 | mm/sec |
| Target mode | Distance | |
| Distance | 13 | mm |
| Calibrate height setting | 35 | mm |
| Bending bridge gap | 10 | mm |
| Bending bridge depth | 25 | mm |

### Angle of Concave Notch Measurement

This measurement is for obtaining the angle between the side proximal edge and the concave notch of the belt part in the contracted state and the extended state.

To obtain the test specimens in the contracted state, a belt part sample (40) is cut open along the side seams to obtain the front belt and/or back belts (84, 86) which comprises a concave notch (108). Cold Spray may be used, paying attention not to make wrinkles in the belt part (40). Care is taken not to spray on any belt elastic member. The obtained belt specimen is laid flat on a hook board in a naturally contracted condition without stretching, and with any wrinkles spread out. Tape may be used to hold the belt specimen in such condition.

To obtain the test specimens in the extended state, the belt part (40) is first subject to the Whole Article Force Measurement for obtaining the Full Stretch Circumference (mm). Then, a belt part sample is cut open along the side seams to obtain the front belt and/or back belts (84, 86) which comprises a concave notch (108). Cold Spray may be used, paying attention not to make wrinkles in the belt part. Care is taken not to spray on any belt elastic member. The obtained belt specimen is stretched to 0.5 times the Full Stretch Circumference and then laid flat on a hook board.

Draw a line between belt specimen proximal edge between one end of side seam to the other (Line 1), then identify the starting position of the concave notch from the side proximal edge (90S). Referring to FIG. 14, draw a line matching the direction of the concave at the starting position (Line 2). If the concave notch takes a curve around the starting position of Line 2, draw a tangent line to the curve at the starting position. Measure the angle (α) between Line 1 and Line 2. Five samples are measured, and their average are calculated and reported to the nearest 1°, respectively.

### EXAMPLES

Example 1 is a pant type absorbent article assembled by a belt part having the primary belt fastening means as a magnet having a polarity and the secondary belt fastening means which comprises loops of a hook-and-loop system, and a crotch part having the primary crotch fastening means as a magnet of opposite polarity as the primary belt fastening means and the secondary crotch fastening means which comprises hooks of a hook-and-loop system. Example 2 is a pant type absorbent article assembled by a belt part having the primary belt fastening means as a combination of opposite magnets having opposite polarity attached to a strap, and the primary crotch fastening means as an opening with the primary belt fastening means. Examples 1 and 2 utilize the magnet fastening system from Fidlock® as the primary fastening system, and are made of the belt part (40) and the crotch part (38) detailed in Tables 1 and 2 below. For both Examples, the crotch part (38) was provided in a folded state using the secondary crotch fastening means along the longitudinal axis. The belt part (40) and the crotch part (38) may be assembled into a pant absorbent article by fastening the primary and secondary fastening means.

TABLE 1

| | | Example 1 | Example 2 |
|---|---|---|---|
| Belt | Plan view | FIGS. 2A and 3A | FIGS. 2C-2D |
| part | | | and 3C-3D |
| | Outer belt | 20 gsm carded nonwoven from Beijing Dayuan | Knitted fabric, |
| | material (92) | Nonwoven Fabric Corp., Ltd | Coolmax ® |
| | Inner belt | 15 gsm spunbond nonwoven from Hubei Gold | made from |
| | material (94) | Dragon Nonwoven Fabric Co., Ltd | polyester fabric |
| | Elastic body | 78 dtex Elastic from HUAFON | NA |
| | (96) | MICROFIBRE(SHANGHAI) Co., Ltd. | |
| | Reinforcement | 78 dtex Elastic from HUAFON | NA |
| | material (40R) | MICROFIBRE(SHANGHAI) Co., Ltd. | |
| | | Provided between outer belt material and | |
| | | 20 gsm carded nonwoven from Beijing Dayuan | |
| | | Nonwoven Fabric Corp., Ltd | |
| | Strap | NA | Woven fabric, |
| | | | Tencel ® made |
| | | | from lyocell |
| | | | fabric |
| | Secondary | 20 gsm spunbond nonwoven from Xiamen | NA |
| | fastening means | Yanjan Industry Co., Ltd. | |
| Crotch | Plan view | FIG. 5A | FIG. 5C |
| part | Topsheet (24) | 40 gsm carded nonwoven from Xiamen Yanjan Industry Co., LTD. | |
| | Backsheet (25) | 14 gsm film from Qifeng new materials Co., Ltd. | |
| | Outer Cover | 25 gsm carded nonwoven from JiangSu Wisdom Nonwoven | |
| | (42) | Technology Co.,Ltd. | |
| | Pocket substrate | 20 gsm spunbond nonwoven from PFNonwovens US Inc. | |
| | (100) | | |
| | Additional | NA | 40 gsm |
| | protruding | | spunbond |
| | material (350) | | nonwoven from |
| | | | PGI |
| | Secondary | 65 gsm hook material from 3M China Co. | NA |
| | fastening means | | |

TABLE 2

| | | Example 1 | Example 2 |
|---|---|---|---|
| Belt part (width mm × height mm) | Front belt | 385 × 110 | 385 × 110 |
| | Back belt | 385 × 130 | 385 × 130 |
| Crotch part (length mm × width mm) | | 428 × 185 | 458 × 261 |
| Secondary Fastening means | Front belt | 275 × 103 | NA |
| (width mm × length mm) | Back belt | 275 × 120 | NA |
| | Front crotch (d1 × d2) one side | 81 × 14 | NA |
| | Back crotch (d1 × d2) one side | 81 × 14 | NA |
| Anchoring zone (length mm) | Front | 63 | NA |
| | Back | 63 | NA |
| Angle of belt notch (degrees) | Front contracted/extended | 90/90 | 90/90 |
| | Back contracted/extended | 90/90 | 90/90 |
| Primary fastening means | T1 (mm) | NA | 173 |
| | T2 (mm) | 20 | 20 |
| | T3 (mm) | NA | 33 |
| | T4 (mm) | NA | 7 |
| | T5 (mm) | 15 | 15 |
| | Strap in open state (mm) | NA | 126 × 26 |
| Crotch part distance between | Front | 50 | 32 |
| bonding region (100B) and proximal | Back | 50 | 41 |
| side contour (100C) along | | | |
| longitudinal axis (mm) | | | |
| Crotch part distance between | Front | 63 | 35 |
| bonding region (100B) and proximal | Back | 101 | 91 |
| side contour (100C) along bonding | | | |
| line (BL) of inner cuff (mm) | | | |

The belt part (40) could be easily donned on the wearer and the crotch part (38) in folded state could be easily opened. The belt part (40) and the crotch part (38) for each Example assembled into a pant absorbent article according to the method of the present invention provided basic functions as a pant absorbent article, such as good containment, leakage prevention, and wear comfort.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A pant absorbent article comprising a belt part and a crotch part, the belt part and the crotch part being detachable with each other by a primary fastening system for providing the absorbent article, the absorbent article having a longitudinal direction and a transverse direction,
the belt part having a front belt and a back belt, the belt part having elasticity in the transverse direction and comprising a pair of side seams having a seam length LS, the front belt and the back belt each comprising a primary belt fastening means on a garment facing side, the primary belt fastening means forming part of the fastening system engageable with a primary crotch fastening means;
the crotch part comprising a water permeable topsheet, a water impermeable backsheet, an absorbent core disposed between the topsheet and the backsheet, and the primary crotch fastening means disposed on the wearer facing side,
wherein the primary belt fastening means and the primary crotch fastening means are fastened magnetically.

2. The pant absorbent article of claim 1, wherein the primary belt fastening means comprises a magnet having a polarity, and the primary crotch fastening means comprises a magnet of opposite polarity of the primary belt fastening means.

3. The pant absorbent article of claim 2, wherein the primary belt fastening means is provided on both the front belt and the back belt, and the primary crotch fastening means is provided on corresponding positions on front and back sides.

4. The pant absorbent article of claim 2, wherein the primary belt fastening means is comprises one or a plurality of magnet pieces, the primary crotch fastening means comprises the same quantity of magnet pieces as the primary belt fastening means, wherein the primary belt fastening means comprises a center belt magnet piece positioned on a longitudinal centerline of the pant absorbent article, and the primary crotch fastening means comprises a center crotch magnet piece positioned on a longitudinal axis of the pant absorbent article.

5. The pant absorbent article of claim 4, wherein the primary crotch fastening means further comprises a pair of side crotch magnet pieces positioned on left and right side edges of the crotch part, and the primary belt fastening means further comprises a pair of side belt crotch magnet pieces in corresponding positions of the side crotch magnet pieces.

6. The pant absorbent article of claim 5, wherein the plurality of magnet pieces on the crotch part all have the same polarity.

7. The pant absorbent article of claim 5, wherein the plurality of magnet pieces on the right side and the left side of the crotch part are provided in opposite polarity.

8. The pant absorbent article of claim 1, wherein the primary belt fastening means comprises a combination of opposite magnets having opposite polarity, and the primary crotch fastening means comprises an engaging means to engage with the primary belt fastening means.

9. The pant absorbent article of claim 8, wherein the primary belt fastening means further comprises a strap which enables engaging with the engaging means for effecting the combination of opposite magnets to attach to each other.

10. The pant absorbent article of claim 9, wherein the engaging means comprises an opening through which the strap is movable.

11. The pant absorbent article of claim 10, wherein the opening is positioned on the left and right side edges of the crotch part, and the primary belt fastening means comprises a pair of side belt opposite magnet combinations in corresponding positions of the side crotch engaging means.

12. The pant absorbent article of claim 11, wherein the magnet pieces from the left and right side belt opposite magnet combinations which are positioned closest to each other are positioned at a distance of T1, wherein T1 is at least about 50 mm.

13. The pant absorbent article of claim 11, wherein each magnet of the opposite magnet combination is substantially a circle and the magnet having a greater diameter has a diameter of T2, wherein the opening to engage with the opposite magnet combination is substantially a circle having a diameter of T3, wherein the following relationship is met: T2<T3<T2×1.5.

14. The pant absorbent article of claim 11, wherein the crotch part comprises an edge elastic element positioned adjacent the front or back edge of the crotch part and extending in the transverse direction.

15. The pant absorbent article of claim 10, wherein the primary belt fastening means comprises a center belt opposite magnet combination positioned on a longitudinal axis, and the primary crotch fastening means comprises a center crotch opening positioned on the longitudinal axis.

16. The pant absorbent article of claim 9, wherein the strap is made of substantially non-elastic material selected from the group consisting of cotton, linen, wool, bamboo, hemp, silk, rayon, polyester, nylon, and combinations thereof.

17. The pant absorbent article of claim 1, wherein the primary fastening system comprises a locking means for maintaining the primary belt fastening means and the primary crotch fastening means in an intended position, the locking means selected from the group consisting of a slit, a rotating means, or combination thereof.

18. The pant absorbent article of claim 1, wherein the belt part is made of durable material selected from the group consisting of cotton, linen, wool, bamboo, hemp, silk, rayon, polyester, nylon, spandex, and combinations thereof.

19. The pant absorbent article of claim 1, further comprising a secondary fastening system.

20. The pant absorbent article of claim 19, wherein the secondary fastening system comprises a hook and loop fastening system, wherein a secondary crotch fastening means comprises hooks, and a secondary belt fastening means comprises loops.
